(19) United States
(12) Patent Application Publication
(10) Pub. No.: US 2007/0071689 A1
(43) Pub. Date: Mar. 29, 2007

(54) Advantageous combination for inhalation of nacystelyn and bronchodilators

(75) Inventors: Arthur M. Deboeck, Gurabo, PR (US); Philippe Baudier, Uccle (BE); Francis Vanderbist, Beersel (BE)

Correspondence Address: LOWE HAUPTMAN BERNER, LLP, 1700 DIAGONAL ROAD, SUITE 300, ALEXANDRIA, VA, 22314, US

(73) Assignee: GALEPHAR M/F, Marche-en-Famenne (BE)

(21) Appl. No.: 10/567,408

(22) PCT Filed: Aug. 6, 2003
(86) PCT No.: PCT/BE03/00134

§ 371 Date: Oct. 5, 2006

(51) Int. Cl. A61K 38/04; A61K 31/4745; A61K 31/137

(52) U.S. Cl. 424/46; 514/19; 514/649; 514/291

(57) ABSTRACT

pharmaceutical combination or composition for inhalation a fixed combination (A) L-lysine N-acetylcysteinate and (B) a bronchodilator agent for simultaneous, sequential or separate administration by inhalation in the treatment of an inflammatory or obstructive respiratory disease.

The present invention provides with advantageous combinations for inhalation containing (A) L-Lysine-N-acetylcysteinate and (B) a bronchodilator agent and possibly pharmaceutical excipients. The composition can be formulated as a nebulizer, a metered dose inhaler or a dry powder inhaler.

### BACKGROUND OF THE INVENTION

The main chronic respiratory diseases are Chronic obstructive pulmonary disease (COPD), asthma and Cystic Fibrosis (CF)

The definition of COPD originating from the Merck Manual of Diagnosis and Therapy, published by Merck Research Laboratories, Division of Merck & Co., Inc., Whitehouse Station, N.J., seventeen edition, 1999 is the following:

A disease characterized by chronic bronchitis or emphysema and airflow obstruction that is generally progressive, may be accompanied by airway hyperreactivity, and may be partially reversible.

On the other hand, asthma is characterized by airway inflammation that is manifested by airway hyperresponsiveness to a variety of stimuli and by airway obstruction that is reversible spontaneously or in response to treatment; reversibility may be incomplete in some patients.

Cystic fibrosis is another chronic inflammatory disease defined as follows :An inherited disease of the exocrine glands, primarily affecting the GI and respiratory systems, and usually characterized by COPD, exocrine pancreatic insufficiency, and abnormally high sweat electrolytes.

The various terms used to quantify the pulmonary functions are described hereinbelow (Quanjer and al., Eur. Respir. J., 1993, 6, Suppl. 16, 5-40, Official Statement of the European Respiratory Society)

Forced Vital Capacity (FVC): is the volume of gas delivered during an expiration made as forcefully and completely as possible starting from full inspiration Forced expiratory volume in one second (FEV1): is the volume of gas exhaled in a one second from the start of the forced vital capacity manoeuvre Peak Expiratory Flow Rate(PEFR): is the maximal flow during a forced expiratory vital capacity manoeuvre starting from a position of full inspiration

Chronic obstructive pulmonary disease (COPD), which includes chronic bronchiti and emphysema, is steadily increasing in frequency, possibly due to continued smoking, increasing air pollution, and the continued ageing of the population.

Prevalence: in the Global Burden of Disease Study conducted under the auspices of the WHO and the World Bank. The world-wide prevalence of COPD in 1990 was estimated to be 9.34/1,000 in men and 7.33/1,000 in women. However, these estimates include all ages and underestimate the true prevalence of COPD in adults. The prevalence of COPD is highest in countries where cigarette smoking has been, or still is, very common, while the prevalence is lowest in countries where smoking is less common, or total tobacco consumption per individual is low.

Morbidity: the limited data that are available indicate that morbidity due to COPD increases with age and is greater in men than women, COPD is responsible for a significant part of physician visits, emergency department visits and hospitalizations.

Mortality: COPD is currently the fourth leading cause of death in the world, and further increases in the prevalence and mortality of the disease can be predicted in the coming decades. In the US, COPD death rates are very low among people under age 45 but then increase with age, and COPD becomes the fourth or fifth leading cause of death among those over 45.

COPD is characterized by edema (oedema) of the mucous membrane, which lines the interior walls of the tracheobronchial tree. When the mucosa accumulates an abnormal quantity of liquid, the profuse and thickened serous fluid excreted may seriously affect ventilation in the alveoli. The mucus resists movement up the walls of the tracheobronchial tree, normally efficiently accomplished by the cilia throughout the airways. Consequently, the serous fluid can form mucus plugs, which can shut off alveoli or an entire airway depriving whole sections of the lung of oxygen-rich air.

Plugs of mucus in the tracheobronchial tree may only partially block the flow of air through the bronchioles. This partial blockage can create a turbulent flow of air, which forms bubbles on the surface of mucosa. When there are enough bubbles, they become foam, which can clog airways and dramatically diminish respiration of the capillaries of the lungs.

The obstruction of the bronchi and bronchioles found in COPD is often a severely disabling condition. A wide variety of compounds including oral methylxanthines, oral and inhaled beta-adrenergic agonists, inhaled cromolyn sodium, inhaled anticholinergics, and oral and inhaled corticosteroids have been tested. Despite the existence of these therapeutic tools, a large number of patients are not responsive to these medications or become non-responsive after a prolonged period of treatment. COPD is always accompanied by an important oxidative stress resulting from an oxidant/antioxidant imbalance, an excess of oxidants and/or a depletion of antioxidants. Oxidative stress is thought to play an important role in the pathogenesis of a number of lung diseases, not only through direct injurious effects, but by involvement in the molecular mechanisms that control lung inflammation. A number of studies have shown an increased oxidant burden and consequently increased markers of oxidative stress in the airspaces, breath, blood, and urine in smokers and in patients with COPD. The presence of oxidative stress has important consequences for the pathogenesis of COPD. These include oxidative inactivation of antiproteinases, airspace epithelial injury, increased sequestration of neutrophils in the pulmonary microvasculature, and gene expression of proinflammatory mediators. With regard to the latter, oxidative stress has a role in enhancing the inflammation that occurs in smokers and patients with COPD, through the activation of redox-sensitive transcription factors such as nuclear factor-κB and activator protein-1, which regulate the genes for proinflammatory mediators and protective antioxidant gene expression.

Asthma is the most common form of bronchoconstrictive disease, which is completely different from COPD. Pathologically, asthma involves constriction of the bronchioles, hypertrophy of the muscles of the bronchioles, and a characteristic infiltrate of eosinophils.

Asthma is the third leading cause of preventable hospitalization in United States. There are about 470.000 hospitalizations and more than 5.000 deaths a year from asthma. Asthma causes recurring episodes of coughing, wheezing, chest tightness, and difficult breathing. Asthma attacks can be life threatening. They can be prevented. Asthma is a chronic inflammatory disorder of the airways. Chronically inflamed airways are hyperresponsive; they become obstructed and airflow is limited (by bronchoconstriction, mucus plugs, and increased inflammation) when airways are exposed to various stimuli, or triggers.

Common asthma triggers (that is, factors that make asthma worse) include viral infections; allergens such as domestic dust mites (in bedding, carpets, and fabric-upholstered furnishings), animals with fur, cockroach, pollens, and molds; tobacco smoke; air pollution, exercise; strong emotional expressions; chemical irritants, and drugs (aspirin and beta blockers).

Asthma attacks (or exacerbations) are episodic, but airway inflammation is chronically present. Asthma is a chronic disorder requiring long-term management. For many patients, this means taking preventive medication every day.

Asthma can change over time. Asthma can be mild, moderate or severe; asthma attacks can be life-threatening. The severity of asthma varies among individuals, and it can change in one individual over time. Treatment decisions are made based on the severity of asthma.

In conclusion, it is clear that all chronic inflammatory diseases of the lungs like COPD and asthma present simultaneously important inflammation and/or oxidation phenomenons of the lung tissue and a significant bronchoconstriction, making the breathing of patients very difficult. No efficient pharmaceutical treatment is currently available to threat all those symptoms simultaneously

A number of patents relating to the treatment of COPD or other pathologies of the respiratory tract have already been granted

The U.S. Pat. No. 6,153,187 describes a method of managing a patient having an accumulation of mucoid, mucopurulent or purulent material containing glycosaminoglycans, the method comprising the step of administering at least one glycosaminoglycon degrading enzyme to the patient in an amount therapeutically effective to reduce at least one of the following: the visco-elasticity of the material, pathogens infectivity and inflammation.

The U.S. Pat. No. 5,969,421 describes a method for prevention of cell death, and method comprising simultaneously topically applying ACC and levulose as an application in an amount effective to result in a synergistic action protecting cells, and presenting cell damage caused by various agents. A pharmaceutical preparation including a combination of ACC and levulose.

The WO Patent 9635452 describes a pharmaceutical composition useful in the treatment of respiratory tract disorders. The composition comprises as active ingredients: (a) acetylcysteine, carbocysteine, erdocysteine or a pharmaceutically acceptable salt of any of theses; and (b) a beta 2 agonist, e.g. salbutamol, terbutaline; and (c) an expectorant e.g. guaiphenesin, sodium citrate, ammonium chloride.

The WO Patent 0010598 describes a method of treating mucus hypersecretion, the causative factor in COPD, asthma and other clinical conditions involving COPD, comprises administering a compound that inhibits exocytosis in mucus secreting cells or neurons that control or direct mucus secretion.

Some inventions describe the combination of a β2 mimetics with an inhaled corticosteroid for the treatment of asthma and COPD. Examples of such inventions are EP416950, which describes the combination of salmeterol with beclomethasone; EP416951 relating to the combination of salmeterol with fluticasone or U.S. Pat. No. 5,674,860 which relates to the combinations of formoterol and budesonide.

L-Lysine N-Acetylcysteinate (also called Nacystelyn or NAL) (U.S. Pat. No. 4,847,282) is an active ingredient efficient in the treatment of chronic inflammatory diseases such as, but not limited to cystic fibrosis (CF) and chronic obstructive pulmonary disease (COPD) (Am. J. Respir. 2000, 161(3)-A72, pediatric pulmonology 22, 161-166 (1996), Free Rad. Biol. Med. 2002, 32(6), 492-502) NAL is actually a water-soluble salt of acetylcysteine (ACC), a widely used mucolytic. A number of experiments have demonstrated that NAL presents several advantages over ACC. The most important of them being the almost neutral pH of NAL (pH ±6.5) what makes its administration possible via the inhalation route without observing any bronchospasms, contrary to ACC, which presents an acidic pH (pH ±2.2) that can be responsible for reflex bronchospasms. This property allows the safe administration of NAL to patients by inhalation via all the inhaled technologies i.e.: nebulization, metered dose inhalers (MDIs) or drug powder inhalers, without the need of any buffering agents (Eur.Respir. J., 1994, 7(1), 81-87.

L-lysine N-acetylcysteinate (CAS N^{o}89344-48-9) has been patented several years ago (U.S. Pat. No. 4,847,282, “Mucolytic acetylcysteine salts”). The principle of the invention was the synthesis of a water soluble acetylcysteine salt, consisting of reaction products of acetylcysteine with at least one basic amino-acid, the latter being preferably selected from the group comprising arginine, lysine, histidine, ornithine and glycine. A number of experiments have been performed to demonstrate that NAL presents several advantages over ACC, its parent molecule: the most important advantage is that NAL has an almost neutral pH (pH=6.5), what makes its administration possible via the inhalation route without observing any side effects (bronchospasms), contrary to ACC which is acidic (pH 2.2).

This allows the safe administration of NAL by inhalation by every galenical form available for this route e.g. nebulization, Metered Dose Inhalers (MDI) and Dry Powder Inhalers (DPI).

A number of experiments have been performed to assess the mucolytic, antioxidant and antiinflammatory properties of NAL in comparison to ACC. It is also important to mention that NAL is active at lower doses than ACC what makes its administration possible by inhalation via portable inhaler devices (DPI or MDI device).

This also greatly facilitates the administration for the patient and hence the compliance since there is no need to use a cumbersome nebulizer which requires an administration time of 10 to 30 minutes, what is difficult to accept for ambulatory patients like the majority of COPD or asthma patients. When NAL is formulated as a dry powder inhaler using a capsule device, the mean administration time for the patient is between 30 and 60 seconds.

The possibility of using a very convenient device and allowing to reach a high lung deposition of NAL is also very advantageous for the treatment of an ambulatory disease like COPD and asthma.

In the past, it has been demonstrated that surprisingly NAL presents a mucolytic and antioxidant activity superior to the activity of ACC (Vanderbist and al Arzneim-Forschung/Drug Research (II) N^{o}8, 1996, Nagy and al., Pulmonary Pharmacology 2 Therapeutics (1997) 10, 287-292, Gillissen and al Respiratory Medicine (1997), 91, 159-168, Tomkiewicz and al. Pulmonary Pharmacology (1995) 8, 259-265, Marriott and al, Eur. Resp. J., 6 (suppl. 17), 438 (5) (abstract), 1993.

For some properties (mucolytic), the superiority of NAL may be explained while for other (antioxidant and antiinflammatory) effects, we can only make some hypothesis about the potential mode of action of the molecule.

Those mucolytic, antioxidant and antiinflammatory properties of NAL result in significant clinical improvement in patients. For instance, it has been demonstrated in cystic fibrosis patients that the administration of NAL can significantly decrease the number and severity of lung exacerbations in comparison to placebo.

Bronchodilators are a very widely used treatment of chronic respiratory diseases. Although they can act through various mechanisms of action, they all result in the dilatation of the airways of the respiratory tract, resulting in an improvement of patient's lung functions such as Forced Vital Capacity (FVC), Forced Expiratory Volume in one second (FEV1) and Peak Expiratory Flow Rate (PEFR). There are two main classes of bronchodilators: the sympaticomimetics, including the short-acting and the long-acting β2-mimetics, and the anticholinergics.

Short-action β2-mimetics include (but are restricted to) salbutamol, terbutaline, fenoterol, pirbuterol or tulobuterol. Each active ingredient cited can be used as the base or as an acceptable pharmaceutical salt. The long-acting β2 mimetics include (but are not restricted to) formoterol and salmeterol. Each molecule can be used as base or as an acceptable pharmaceutical salt. The anticholinergic drugs include (but are not restricted to) ipratropium, oxitropium or tiotropium.

In general, bronchodilators are used in asthma and chronic obstructive pulmonary diseases but can be administered in almost every inflammatory disease of the lung. Never described was a combination for inhalation of the mucoactive agent L-lysine N-acetylcysteinate (Nacystelyn or NAL) with a bronchodilator agent.

### FIELD OF THE INVENTION

Chronic respiratory diseases involve complexes mechanisms and require the adminsitration of different kinds of treatment to patients among with antibiotics, corticosteroids, bronchodilators, mucolytics, . . . . The field of the present invention is to provide an advantageous, efficient and safe treatment of such diseases by administering an combination for inhalation comprising the mucoactive agent Nacystelyn and at least one bronchodilator agent. Advantageous combinations are disclosed in the attached claims.

It has now suprisingly been found that NAL and bronchodilators can advantageously be combined to be used by inhalation in the treatment of chronic respiratory diseases such as cystic fibrosis, chronic obstructive pulmonary disease or asthma. NAL has a duration of action that makes its administration possible from 1 to 4 times a day. The bronchodilator class of molecules include molecules with very long duration of action which have to be administered only once a day (tiotropium) but also long acting β2-mimetics which are usually administered twice a day like formoterol and salmeterol. Finally there are short-acting bronchodilators such as salbutamol, terbutaline, ipratropium or oxitropium which have to be administered 4 to 6 times a day. Consequently, NAL can be combined in a single pharmaceutical form with every type of bronchodilators. NAL and bronchodilators offer complementary modes of action which make their administration particularly profitable for the patients. Indeed, NAL, through its mucolytic activity may allow to the bronchodilator to be deposited more deeply in the lungs after inhalation i.e. in its target tissue, the bronchi and the bronchioli, so permitting a better efficacy of the bronchodilator agent. On the other hand, through its action of dilatation of the conducting airways, the bronchodilator agent will allow to NAL to also be deposited deeply in the lungs so guarantying an optimal antiinflammatory and antioxydant of NAL, all along the respiratory tract.

Using such combination therapy, medicaments which result in a significant improvement in lung function may be prepared. In another aspect, using the combination therapy of the invention, medicaments which provide improved control of obstructive or inflammatory airways diseases, or a reduction in exacerbation's of such diseases, may be prepared. In a further aspect, using compositions of the invention, medicaments which can be used on demand in rescue treatment of obstructive or inflammatory airways diseases, or which reduce or eliminate the need for treatment with short-acting rescue medicaments such as salbutamol or terbutaline, may be prepared; thus medicaments based on compositions of the invention facilitate the treatment of an obstructive or inflammatory airways disease with a single medicament.”

The combination allowing an administration twice or once a day will be preferred to combinations requiring more administrations because they increase the comfort and the compliance of the patients. NAL and the bronchodilator can be administered simultaneously, separately or sequentially. The combination of NAL and the bronchodilator agent can be formulated as a nebulizer, as metered dose inhalers (MDI) or as a drug powder inhaler (DPI).

For a more easier understanding of which kind of combination the present invention is relating, in the following of the present text, the letter (A) will be used to design Nacystelyn and the letter (B) will be used to design the bronchodilator agent. The present invention can for instance, consist in the administration of the medicament or pharmaceutical composition as hereinbefore described, i.e. with (A) and (B) in admixture or separate, by inhalation, i.e. (A) and (B) or the mixture thereof are in inhalable form. The inhalable form of the medicament i.e. of (A) and (B) may be, for example, an atomizable composition such as an aerosol comprising the active ingredient, i.e. (A) and (B) separately or in admixture, in solution or dispersion in a propellant, or a nebulizable composition comprising a dispersion of the active ingredient in an aqueous, organic or aqueous/organic medium. For example, the inhalable form (suitable for inhalation) of the medicament may be an aerosol comprising a mixture of (A) and (B) in solution or dispersion in a propellant, or a combination of an aerosol containing (A) in solution or dispersion in a propellant with an aerosol containing (B) in solution or dispersion in a propellant. In another example, the inhalable form is a nebulizable composition comprising a dispersion of (A) and (B) in an aqueous, organic or aqueous/organic medium, or a combination of a dispersion of (A) in such a medium with a dispersion of (B) in such a medium.

An aerosol composition suitable for use as the inhalable form of the medicament may comprise the active ingredient in solution or dispersion in a propellant, which may be chosen from any of the propellants known in the art. Suitable such propellants include hydrocarbons such as n-propane, n-butane or isobutane or mixtures of two or more such hydrocarbons, and halogen-substituted hydrocarbons, for example fluorine-substituted methanes, ethanes, propanes, butanes, cyclopropanes or cyclobutanes, particularly 1,1,1,2-tetrafluoroethane (HFA134a) and 1,1,1,2,3,3,3-heptafluoropropane (HFA227), or mixtures of two or more such halogen-substituted hydrocarbons. Where the active ingredient is present in suspension in the propellant, i.e. where it is present in particulate form dispersed in the propellant, the aerosol composition may also contain a lubricant and a surfactant, which may be chosen from those lubricants and surfactants known in the art, like for instance sorbitan oleate and derivatives. Other suitable aerosol compositions include surfactant-free or substantially surfactant-free aerosol compositions. The aerosol composition may contain up to about 5% by weight, for example 0.002 to 5%, 0.01 to 3%, 0.015 to 2%, 0.1 to 2%, 0.5 to 2% or 0.5 to 1%, by weight of the active ingredients, based on the weight of the propellant. Where present, the lubricant and surfactant may be in an amount up to 5% and 0.5% respectively by weight of the aerosol composition. The aerosol composition may also contain a co-solvent such as ethanol in an amount up to 30% by weight of the composition, particularly for administration from a pressurized metered dose inhalation device.

In another embodiment of the invention, the inhalable form is a dry powder, i.e. (A) and (B) are present in a dry powder comprising finely divided (A) and (B) optionally together with a finely divided pharmaceutically acceptable carrier, which is preferably present and may be one or more materials known as pharmaceutically acceptable carriers, preferably chosen from materials known as carriers in dry powder inhalation compositions, for example saccharides, including monosaccharides, disaccharides, polysaccharides and sugar alcohols such as arabinose, glucose, fructose, ribose, mannose, sucrose, trehalose, lactose, maltose, starches, dextran or mannitol. An especially preferred carrier is lactose. The dry powder may be in capsules of hard gelatin or hydroxypropylmethylcellulose, or in blisters, for use in a dry powder inhalation device, preferably in dosage units of (A) and (B) together with the carrier in amounts to bring the total weight of powder per dose to from 5 mg to 50 mg. Alternatively, the dry powder may be contained as a reservoir in a multi-dose dry powder inhalation device.

In the finely divided particulate form of the medicament, and in the aerosol composition where the active ingredient is present in particulate form, the active ingredient may have an average particle diameter of up to about 10 μm, for example 0.1 to 5 μm, preferably 1 to 5 μm. The solid carrier, where present, generally has a maximum particle diameter up to 300 μm, preferably up to 212 μm, and conveniently has a mean particle diameter of 100 to 160 μm, e.g. 100 to 125 μm. The particle size of the active ingredient, and that of a solid carrier where present in dry powder compositions, can be reduced to the desired level by conventional methods, for example by grinding in an air-jet mill, ball mill or vibrator mill, microprecipitation, spray-drying, lyophilisation or recrystallisation from supercritical media. the carrier material can also consist in a mix of different materials in order to optimize the properties of the dry powder inhaler formulation. The inhalable medicament may be administered using an inhalation device suitable for the inhalable form, such devices being well known in the art. Accordingly, the invention also provides a pharmaceutical product comprising a medicament or pharmaceutical composition as hereinbefore described in inhalable form as hereinbefore described in association with one or more inhalation devices. In a further aspect, the invention provides an inhalation device, or a pack of two or more inhalation devices, containing a medicament or pharmaceutical composition as hereinbefore described in inhalable form as hereinbefore described.

### EXAMPLES

The invention is additionally illustrated in connection with the following examples, which are considered to be illustrative of the present invention. It should be understood, however, that the invention is not limited to the specific details of the Examples.

### Example 1

### Combination: NAL/Ipratropium 50/0.25 mg—Powder for Nebulization

Formula Per Vial:

Nacystelyn: 50 mg

ipratropium bromide: 0.25 mg

Mannitol: 44.7 mg

sodium edatate (EDTA): 0.10 mg

reconstitution is made with 2 ml of deionized water (degassed for 30 minutes)

Process: The combination was manufactured using a freeze-drying process. (freeze-drying machine: GT2 (leybold-Heraeus). The solution of Nacystelin and ipratropium bromide (in a solvent, advantageously in an aqueous solvent or water containing solvent) with mannitol was filled (1 ml) into Typr I glass vials (Gaasch packaging) under constant nitrogen gas bubbling. The vials were gassed with nitrogen and partly closed with rubber stoppers (FM257/2, Helvoet Pharma) and placed inside the Freeze-dryer. After lyophilization, the chalber was vented with nitrogen gas and the vials were automatically closed and sealed with Alcaps.

The so obtained powder is then suitable for ensuring a nebulization of the compounds with an aqueous medium.

### Example 2

### NAL/Oxitropium Bromide 4/0.1 mg/Puff

Formula

| ingredient | Amount/puff (mg/puff) |
|---|---|
| Nacystelyn | 2.0 |
| Oxitropium bromide | 0.1 |
| lecithin | 7.5 |
| monofluorotrichloroethane | 25.0 |
| difluorodichloromethane | 60.0 |

### Example 3

### NAL/Salmeterol 3/0.050 mg/Puff

The salmeterol used was under the form of salmeterol xinafoate. 73.2 mg of salmeterol xinafoate are equivalent to 50.0 mg of salmeterol base

Formula:

| ingredient | Amount/puff (mg/puff) |
|---|---|
| Nacystelyn | 4 |
| salmeterol xinafoate | 0.073 |
| lecithin | 7.5 |
| monofluorotrichloroethane | 20.0 |
| difluorodichloromethan | 50.0 |

### Example 4

### Combination NAL/Formoterol—Dry Powder Inhaler Formulation

Formula:

| ingredient | Amount/dose (mg/dose) |
|---|---|
| Nacystelyn | 4.0 |
| Formoterol fumarate | 0.006 |
| Lactose monohydrate | 5.90 |

Process: formoterol fumarate (with a weight average particle size comprised between 1 and 5 μm) is pre-blended with a small fraction of lactose ( 1/10 of the total amount of lactose with a weight particle size comprised between 100 and 300 μm, such as about 160 μm,) in a cubic mixer (Turbula). NAL (with a weight average particle size comprised between 1 and 5 μm) is blended in a planetary mixer (Colette) with the remaining part of lactose ( 9/10 of the amount of lactose) for 10 minutes on speed 1. The pre-blend formoterol/lactose is then added to the NAL/lactose mix in the planetary mixer (sandwich mix) and the final blend is mix at speed 1 for another 10 minutes. The resulting powder is then manually filled into Multidose DPI device (Mulidose MIAT Inhaler, Milano, Italy). Another part of the mix has been filled into size 3 hydroxypropylmethylcellulose capsules to be used with a single dose DPI device (Miat Monodose inhaler).

### Example 5

### Combination NAL/Formoterol Fumarate—Dry Powder Inhaler Formulation 10/0.012 mg

Formula:

| ingredient | Amount/capsule (mg/capsule) |
|---|---|
| Nacystelyn | 10.0 |
| Formoterol fumarate | 0.012 |
| Lactose monohydrate | 8.0 |
| Anhydrous lactose | 22.0 |

The mixing process was the same as in example 4.

The powder was filled into transparent-transparent, size 3 hydroxypropylmethylcellulose capsules and administered with the monodose Miat inhaler (Miat, Milano, Italy).

### Example 6

### Stability Data on NAL/Formoterol DPI from Example 5

Stability data on NAL/formoterol capsules packaged in aluminum/aluminum blisters

A) 25° C./60% RH

| | T0 | T3 Months | T6 Months | T12 Months | T18 Months | T24 Months |
|---|---|---|---|---|---|---|
| Assay | | | | | | |
| formoterol (%) | 99.8 | 98.9 | 99.6 | 99.2 | 99.1 | 99.3 |
| NAL (%) | 101.2 | 100.6 | 100.4 | 100.7 | 100.8 | 100.2 |
| Impurities | | | | | | |
| a) formoterol | | | | | | |
| impurity 52008RC01 (%) | 0.2 | 0.3 | 0.2 | 0.3 | 0.4 | 0.5 |
| total impurities (%) | 0.2 | 0.3 | 0.3 | 0.4 | 0.5 | 0.6 |
| b) NAL | | | | | | |
| NN-diaceylcystine (%) | 0.15 | 0.20 | 0.22 | 0.30 | 0.32 | 0.46 |
| total impurites (%) | 0.20 | 0.22 | 0.25 | 0.26 | 0.27 | 0.28 |
| Fine particle Dose | | | | | | |
| formoterol (μg) | 3.7 | 3.6 | 3.8 | 3.6 | 3.9 | 3.8 |
| NAL (mg) | 3.2 | 3.1 | 3.1 | 3.0 | 3.2 | 3.1 |

The stability tests were carried out in order to establish the presence of impurities and the fine particle dose after preparation, as well as after a storage period of 3 months, 6 months, 12 months, 18 months and 24 months at 25° C. with a relative humidity of 60%. This test shows the stability of the composition with respect to the formation of impurities, as well as with respect to the fine particle dose for the formoterol and for the NAL.

### Example 7

### Combination NAL/Salmeterol—Dry Powder Inhaler Formulation 10/0.073 mg (Equal to 50.0 mg of Salmeterol Base)

| ingredient | Amount/capsule (mg/capsule) |
|---|---|
| Nacystelyn | 10.0 |
| Salmeterol xinafoate | 0.073 |
| Lactose monohydrate | 8.0 |
| Anhydrous lactose | 22.0 |

### Example 8

### Fine Particle Dose (FPD) Obtained with the DPI Combination NAL/Salmeterol of Example 7 Versus the Marketed DPI Form of Salmeterol DPI (Serevent 50 μg, Diskus)

From an In vitro comparative deposition of salmeterol from (i) the combination NAL/salmeterol 10/0.025 mg of the current invention versus (ii) the Serevent 50 μg, Diskus (Deposition measured with a Multistage Liquid Impinger, 4 liters of air, n=3), it appears that the fine particle dose obtained with the combination of the invention (NAL+25 μg salmetertol) was 10.9 μg salmeterol, while said fine particle dose was 9.89 μg salmeterol for the Serevent 50 μg salmeterol

1. A pharmaceutical combination or composition for inhalation a fixed combination (A) L-lysine N-acetylcysteinate and (B) a bronchodilator agent for simultaneous, sequential or separate administration by inhalation in the treatment of an inflammatory or obstructive respiratory disease.

2. The composition or combination of claim 1, wherein the bronchodilator agent belongs to the class of β2-mimetics.

3. The composition or combination of claim 1, wherein the bronchodilator agent belongs to the class of long-acting β2-mimetics.

4. The composition or combination of claim 3, wherein the bronchodilator agent is formoterol or a solvate thereof, or salmeterol or a salt thereof, or mixtures thereof.

5. The composition or combination of claim 2, wherein the bronchodilator agent is a short-acting β2-mimetic such as but not limited to salbutamol, terbutaline, pirbuterol, fenoterol, tulobuterol, and mixtures thereof.

6. The composition or combination of claim 1, wherein the bronchodilator agent belong to the class of anticholinergics.

7. The composition or combination of claim 6, wherein the bronchodilator agent is tiotropium, oxitropium, ipratropium and mixtures thereof.

8. The composition or combination of claim 1, wherein the weight ratio of (A) to (B) is from 1:10 to 1:10 000, preferably from 1:100 to 1:2000, most preferably from 1:200 to 1:1000.

9. The composition or combination of claim 1, wherein the composition or combination contained in addition of (A) and (B) at least one pharmaceutically acceptable carrier.

10. The composition or combination of claim 1 suitable for an inhalable dry powder inhaler comprising micronized particles of (A) L-lysine N-acetylcysteinate and (B) micronized particles of (B) a bronchodilator agent, advantageously mixed with one or more acceptable excipients(s)

11. The composition or combination of claim 10, wherein the composition or combination is in a form suitable for inhalation through a monodose inhaler device.

12. The composition or combination of claim 10, wherein the composition or combination is in a form suitable for inhalation through a multiple-dose inhaler.

13. The composition or combination of claim 10, containing in addition to (A) and (B) at least one saccharide.

14. The composition or combination of claim 13, where the saccharide is lactose.

15. The composition or combination of claim 1, wherein the composition or combination is an inhalable pressurized metered dose inhaler.

16. The composition or combination of claim 15, wherein the composition or combination contains in addition to (A) and (B) at least a propellant wherein (A) and (B) are either dissolved or dispersed.

17. The composition or combination of claim 15, wherein the propellant is chlorofluorocarbon derivative.

18. The composition or combination of claim 15, where the propellant is a hydrofluorocarbon derivative.

19. The composition or combination of claim 1, wherein the composition or combination is an inhalable nebulizable composition or combination comprising a solution and/or a dispersion of (A) and (B) in an aqueous medium.

20. The composition or combination of claim 1, which is a dry powder inhaler in a capsule containing from 2 to 20 μg of L-lysine N-acetylcysteinate and from 2 to 20 μg of formoterol or a solvate thereof.

21. The composition or combination of claim 1, which is a dry powder inhaler in a capsule containing from 2 to 20 μg of L-lysine N-acetylcysteinate and from 10 to 100 μg of salmeterol or a solvate thereof.

22. The composition or combination of claim 1, which is a dry powder inhaler in a capsule containing from 2 to 20 μg of L-lysine N-acetylcysteinate and from 10 to 100 μg of tiotropium or a solvate thereof.

23. A method of treating an inflammatory or obstructive respiratory disease which comprises administering to a subject in need of such treatment effective amounts of (A), as defined in claim 1, and (B) as defined in claim 1.
